SEQUENCE LISTING (31 sequences)

SEQ ID NO: 1 - RNA, 1477 nt, Staphylococcus gallinarum
aggaugaacgcuggcggcgugccuaauacaugcaagucgagcgaacagauaaggagcuugcuccuuugacguuagcggcggacgggugaguaacacguggguaaccuaccuauaagacuggaauaacuccgggaaaccggggcuaaugccggauaacauauagaaccgcaugguucuauagugaaagaugguuuugcuaucacuuauagauggacccgcgccguauuagcuaguugguaagguaauggcuuaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaaggguuucggcucguaaaacucuguuauuagggaagaacauauguguaaguaacugugcacaucuugacgguaccuaaucagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaccacucuagagauagagcuuuccccuucgggggacaaagugacagguggugcaugguugucgucagcucgugucgugagauguuggguuaagucccgcaacgagcgcaacccuuaagcuuaguugccaucauuaaguugggcacucuagguugacugccggugacaaaccggaggaagguggggaugacgucaaaucaucaugccccuuaugauuugggcuacacacgugcuacaauggacaauacaaagggcagcuaaaccgcgaggucaugcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauuggggug

SEQ ID NO: 2 - RNA, 1493 nt, Staphylococcus delphini
agaguuugauccuggcucaguaugaacgcuggcggcgugccuaauacaugcaagucgagcgaacagauaaggagcuugcuccuuugacguuagcggcggacgggugaguaacacguggguaaccuaccuauaagacuggaauaacuccgggaaaccggggcuaaugccggauaacauguugaaccgcaugguucuacagugaaagacggucuugcugucacuuauagauggacccgcgccguauuagcuaguuggugggguaacggccuaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaagcucuguuguuagggaagaacaaauguguaaguaacugugcacaucuugacgguaccuaaccagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcggcuuucuggucugcaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacggucgcaagacugaaacucaaaggaauugacggggacccgcacaagcgguggagcaugugguuuaauucgaagcaacgcgaagaaccuuaccaaaucuugacauccuuugacaacucuagagauagagcuuuccucuucggaggacaaagugacagguggugcaugguugucgucagcucgugucgugagauguuggguuaagucccgcaacgagcgcaacccuugaacuuaguugccaucauuaaguugggcacucuaaguugacugccggugacaaaccggaggaagguggggaugacgucaaaucaucaugccccuuaugauuugggcuacacacgugcuacaauggacaauacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuuggagcuagccgucgaaggugggacaaaugauuggggu

SEQ ID NO: 3 - RNA, 1470 nt, Staphylococcus warneri
gaugaacgcuggcggcgugccuaauacaugcaagucgagcgaacagauaaggagcuugcuccuuugacguuagcggcggacgggugaguaacacguggauaaccuaccuauaagacugggauaacuucgggaaaccggagcuaauaccggauaacauauugaaccgcaugguucaauagugaaaggcggcuuugcugucacuuauagauggauccgcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuaucagggaagaacaaauguguaaguaacugugcacaucuugacgguaccugaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaccgcucuagagauagagucuuccccuucgggggacaaagugacagguggugcaugguugucgucagcucgugucgugagauguuggguuaagucccgcaacgagcgcaacccuuaagcuuaguugccaucauuaaguugggcacucuaaguugacugccggugacaaaccggaggaagguggggaugacgucaaaucaucaugccccuuaugauuugggcuacacacgugcuacaauggacaauacaaagggcagcuaaaccgcgaggucaascaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauug

SEQ ID NO: 4 - RNA, 1553 nt, Staphylococcus saprophyticus
uuuauggagaguuugauccuggcucaggaugaacgcuggcggcgugccuaauacaugcaagucgagcgaacagauaaggagcuugcuccuuugacguuagcggcggacgggugaguaacacguggguaaccuaccuauaagacugggauaacuucgggaaaccggagcuaauaccggauaacauuuggaaccgcaugguucuaaagugaaagaugguuuugcuaucacuuauagauggacccgcgccguauuagcuaguugguaagguaacggcuuaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaaggguuucggcucguaaaacucuguuauuagggaagaacaaauguguaaguaacugugcacgucuugacgguaccuaaucagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaaaacucuagagauagagccuuccccuucgggggacaaagugacagguggugcaugguugucgucagcucgugucgugagauguuggguuaagucccgcaacgagcgcaacccuuaagcuuaguugccaucauuaaguugggcacucuagguugacugccggugacaaaccggaggaagguggggaugacgucaaaucaucaugccccuuaugauuugggcuacacacgugcuacaauggacaauacaaagggcagcuaaaccgcgaggucaugcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauuggggugaagucguaacaagguagccguaucggaaggugcggcuggaucaccuccuuu

SEQ ID NO: 5 - RNA, 1468 nt, Staphylococcus epidermidis
gaugaacgcuggcggcgugccuaauacaugcaagucgagcgaacagacgaggagcuugcuccucugacguuagcggcggacgggugaguaacacguggauaaccuaccuauaagacugggauaacuucgggaaaccggagcuaauaccggauaauauauugaaccgcaugguucaauagugaaagacgguuuugcugucacuuauagauggauccgcgccgcauuagcuaguugguaagguaacggcuuaccaaggcaacgaugcguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuauuagggaagaacaaauguguaaguaacuaugcacgucuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccucugaccccucuagagauagaguuuuccccuucgggggacagagugacagguggugcaugguugucgucagcucgugucgugagauguuggguuaagucccgcaacgagcgcaacccuuaagcuuaguugccaucauuaaguugggcacucuaaguugacugccggugacaaaccggaggaagguggggaugacgucaaaucaucaugccccuuaugauuugggcuacacacgugcuacaauggacaauacaaaggguagcgaaaccgcgaggucaagcaaaucccauaaaguuguucucaguucggauuguagucugcaacucgacuauaugaagcuggaaucgcuaguaaucguagaucagcaugcuacggugaauacguucccgggucuuguacacaccgcccgucacaccacgagaguuuguaacacccgaagccgguggaguaaccauuuggagcuagccgucgaaggugggacaaaugauug

SEQ ID NO: 6 - RNA, 1494 nt, Staphylococcus cohnii
agaguuugauccuggcucaggaugaacgcuggcggcgugccuaauacaugcaagucgagcgaacagauaaggagcuugcuccuuugacguuagcggcggacgggugaguaacacguggguaaccuaccuauaagacuggaauaacuccgggaaaccggggcuaaugccggauaacauuuagaaccgcaugguucuaaagugaaagaugguuuugcuaucacuuauagauggacccgcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuauuagggaagaacaaauguguaaguaacugugcacgucuugacgguaccuaaucagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugacaacucuagagauagagccuuccccuucgggggacaaagugacagguggugcaugguugucgucagcucgugucgugagauguuggguuaagucccgcaacgagcgcaacccuuaagcuuaguugccagcauuaaguugggcacucuaaguugacugccggugacaaaccggaggaagguggggaugacgucaaaucaucaugccccuuaugauuugggcuacacacgugcuacaauggacaauacaaagggcagcuaaaccgcgaggucaugcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauuggggu

SEQ ID NO: 7 - RNA, 1475 nt, Staphylococcus chromogenes
aggaugaacgcuggcggcgugccuaauacaugcaagucgagcgaacugacgaggagcuugcuccuuugacguuagcggcggacgggugaguaacacguggguaaccuaccuauaagacuggaauaacuccgggaaaccggggcuaaugccggauaacauaucgaaccgcaugguucgauagugaaagacggucuugcugucacuuauagauggacccgcgccguauuagcuaguuggugagguaacggcucaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaagcucuguuguuagggaagaacaaauguguaaguaacugugcacaucuugacgguaccuaaccagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcggcuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacggucgcaagacugaaacucaaaggaauugacggggacccgcacaagcgguggagcaugugguuuaauucgaagcaacgcgaagaaccuuaccaaaucuugacaucuuuugaccacucuagagauagaguuuuccucuucggaggacaaaaugacagguggugcaugguugucgucagcucgugucgugagauguuggguuaagucccgcaacgagcgcaacccuugagcuuaguugccaucauuaaguugggcacucuaaguugacugccggugacaaaccggaggaagguggggaugacgucaaaucaucaugccccuuaugauuugggcuacacacgugcuacaauggacaauacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuggagcuagccgucgaaggugggacaaaugauuggggug

SEQ ID NO: 8 - RNA, 1544 nt, Staphylococcus haemolyticus
  misc_feature (12)..(13): n is a, c, g, or u
agaguuugauunnggcucaggaugaacgcuggcggcgugccuaauacaugcaagucgagcgaacagacaaggagcuugcuccuuugacguuagcggcggacgggugaguaacacguggguaaccuaccuauaagacugggauaacuucgggaaaccggagcuaauaccggauaauauuucgaaccgcaugguucgauagugaaagaugguuuugcuaucacuuauagauggacccgcgccguauuagcuaguugguaagguaacggcuuaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuauuagggaagaacauacguguaaguaacuaugcacgucuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuguaaaacuugagugcagaagaggaaaguggaauuccauguguagcggugaaaugcgcagagauauggaggaacaccaguggcgaaggcgacuuucuggucucuaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaaguguaagaaccuuaccaaaucuugacauccuuugacaacucuagagauagagccuuccuucgggggacaaagugacagguggugcaugguugucgucagcucgugucgugagauguuggguuaagucccgcaacgagcgcaacccuuaagcuuaguugccaucauuaaguugggcacucuaaguugacugccggugacaaaccggaggaagguggggaugacgucaaaucaucaugccccuuaugauuugggcuacacacgugcuacaauggacaauacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuggagcuagccgucgaaggugggacaaaugauuggggugaagucguaacaagguagccguaucggaaggugcggcuggaucaccuccuuucu

SEQ ID NO: 9 - RNA, 1480 nt, Staphylococcus lentus
aggaugaacgcuggcggcgugccuaauacaugcaagucgagcgaacagaugagaagcuugcuucucugauguuagcggcggacgggugaguaacacguggguaaccuaccuauaagacugggauaacuccgggaaaccggggcuaauaccggauaauauauugaaccgcaugguucaauguugaaagacgguuucggcugucacuuauagauggacccgcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuuaggaucguaaaacucuguuguuagggaagaacaaauuuguuaguaacugaacaagucuugacgguaccuaaccagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacuggggaacuugagugcagaagaggagaguggaauuccauguguagcggugaaaugcgcagagauauggaggaacaccaguggcgaaggcggcucucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaucgcucuagagauagaguuuuccccuucgggggacaaagugacagguggugcaugguugucgucagcucgugucgugagauguuggguuaagucccgcaacgagcgcaacccuuaagcuuaguugccaucauuaaguugggcacucuagguugacugccggugacaaaccggaggaagguggggaugacgucaaaucaucaugccccuuaugauuugggcuacacacgugcuacaauggauaauacaaagggcagcgaacccgcgaggucaagcaaaucccauaaaauuauucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccuuuuauuaggagcuagccgucgaaggugggacagaugauuggggug

SEQ ID NO: 10 - RNA, 1470 nt, Staphylococcus auricularis
ggaugaacgcuggcggcgugccuaauacaugcaagucgagcgaacagauaaggagcuugcuccuuugacguuagcggcggacgggugaguaacacguggguaaccuaccuauaagacuggaauaacuccgggaaaccggggcuaaugccggauaacauguugaaccgcaugguucuacagugaaaggyggcuuugcugucacuuauagauggacccgcgccguauuagcuaguuggugagguaacggcucaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuauuagggaagaacaaaugcguaaguaacugugcgcgucuugacgguaccuaaucagaaagccacggyuaacuacgugccagcagccgcgguaauacguagguggcaagcguuauccggaauuauugggcguaaagcgcgcguaggcgguuuuuuaagucugaugugaaagcccacggcucaaccguggagggucauuggaaacugaaaaacuugagugcagaagaggaaaguggaauuccauguguagcggugaaaugcgcagagauauggaggaacaccaguggcgaagccgacuuucuggucuguaacugacgcugaugugcgaaagcguggggaucaaacaggauuagauacccugguagucccacgccguaaacgaugagugcuaaguguuaggggguuuccgccccuuagugcuguagcuaacgcauuaagcacuccgccuggggaguacggccgcaaggcuuaaacucaaaggaauugacggggacccgcacaagcgguggagcaugugguuuaauucgaagcaacgcgaagaaccuuaccaaaucuugacauccuuugaccgcucuagagauagagucuuccccuucgggggacaaagugacagguggugcaugguugucgucagcucgugucgugagauguuggguuaagucccgcaacgagcgcaacccuuaagcuuaguugccaucauucaguugggcacucuaaguugacugccggugacaaaccggaggaagguggggaugacgucaaaucaucaugccccuuaugauuugggcuacacacgugcuacgauggacaauacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagucgguagaguaaccuuuuggagcuagccgccgaaggugggacaaaugauug

SEQ ID NO: 11 - RNA, 1494 nt, Staphylococcus equorum
agaguuugauccuggcucaggaugaacgcuggcggcgugccuaauacaugcaagucgagcgaacggauaaggagcuugcuccuuugaaguuagcggcggacgggugaguaacacguggguaaccuaccuauaagacuggaauaacuucgggaaaccggagcuaaugccggauaacauuuggaaccgcaugguucuaaaguaaaagaugguuuugcuaucacuuauagauggacccgcgccguauuagcuaguugguaagguaacggcuuaccaaggcaacgauacguagccgaccugagagggugaucggccacacuggaacugagacacgguccagacuccuacgggaggcagcaguagggaaucuuccgcaauggacgaaagucugacggagcaacgccgcgugagugaugaagguuuucggaucguaaaacucuguuauuagggaagaacaaauguguaaguaacugugcacaucuugacgguaccuaaucagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggaaacuugaguacagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaaaacucuagagauagagccuuccccuucgggggacaaagugacagguggugcaugguugucgucagcucgugucgugagauguuggguuaagucccgcaacgagcgcaacccuuaaacuuaguugccagcauuuaguugggcacucuagguugacugccggugacaaaccggaggaagguggggaugacgucaaaucaucaugccccuuaugauuugggcuacacacgugcuacaauggacaauacaaagggcagcuaaaccgcgaggucaugcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauuggggu

SEQ ID NO: 12 - RNA, 1474 nt, Staphylococcus intermedius
gaugaacgcuggcggcgugccuaauacaugcaagucgagcgaacagauaaggagcuugcuccuuugacguuagcggcggacgggugaguaacacguggguaaccuaccuauaagacuggaauaacuccgggaaaccggggcuaaugccggauaacauguugaaccgcaugguucuacagugaaagacggucuugcugucacuuauagauggacccgcgccguauuagcuaguuggugggguaacggccuaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaagcucuguuguuagggaagaacaaauguguaaguaacugugcacaucuugacgguaccuaaccagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcggcuuucuggucugcaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacggucgcaagacugaaacucaaaggaauugacggggacccgcacaagcgguggagcaugugguuuaauucgaagcaacgcgaagaaccuuaccaaaucuugacauccuuugaccgcucuagagauagaguuuuccucuucggaggacaaagugacagguggugcaugguugucgucagcucgugucgugagauguuggguuaagucccgcaacgagcgcaacccuugaacuuaguugccaucauucaguugggcacucuaaguugacugccggugacaaaccggaggaagguggggaugacgucaaaucaucaugccccuuaugauuugggcuacacacgugcuacaauggacaauacaaagggcagcaaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuuggagcuagccgucgaaggugggacaaaugauuggggug

SEQ ID NO: 13 - RNA, 1494 nt, Staphylococcus pasteuri
agaguuugauccuggcucaggacgaacgcuggcggcgugccuaauacaugcaagucgagcgaacagauaaggagcuugcuccuuugacguuagcggcggacgggugaguaacacguggauaaccuaccuauaagacugggauaacuucgggaaaccggagcuaauaccggauaagauuuugaaccgcaugguucaauagugaaagacggccuugcugucacuuauagauggauccgcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuaucagggaagaacaaacguguaaguaacugugcacgucuugacgguaccugaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaccgcucuagagauagaguuuuccccuucgggggacaaagugacagguggugcaugguugucgucagcucgugucgugagauguuggguuaagucccgcaacgagcgcaacccuuaagcuuaguugccaucauuaaguugggcacucuaaguugacugccggugacaaaccggaggaagguggggaugacgucaaaucaucaugccccuuaugauuugggcuacacacgugcuacaauggacaauacaaagggcagcuaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauuggggu

SEQ ID NO: 14 - RNA, 1537 nt, Staphylococcus muscae
uugauccuggcucaggaugaacgcuggcggcgugccuaauacaugcaagucgagcgaacagacgaggugcuugcaccucugacguuagcggcggacgggugaguaacacguggguaaccuaccuauaagacuggaauaacuucgggaaaccggagcuaaugccggauaauauauugaaccgcaugguucaauagugaaagacgguuuugcugucacuuauagauggacccgcgccguauuagcuaguugguaagguaacggcuuaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaagcucuguuauuagggaagaacaaacguguaaguaacugugcacgucuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcggcuuucuggucugcaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacggucgcaagacugaaacucaaaggaauugacggggacccgcacaagcgguggagcaugugguuuaauucgaagcaacgcgaagaaccuuaccaaaucuugacauccuuugaccgcacuagagauaguguuuuccucuucggaggacaaagugacagguggugcaugguugucgucagcucgugucgugagauguuggguuaagucccgcaacgagcgcaacccuugagcuuaguugccaucauuaaguugggcacucuaaguugacugccggugacaaaccggaggaagguggggaugacgucaaaucaucaugccccuuaugauuugggcuacacacgugcuacaauggacauuacaaagggcagcgaaaccgcgaggucaagcaaaucccauaaagauguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuggagcuagccgucgaaggugggacaaaugauuggggugaagucguaacaagguagccguaucggaaggugcggcuggaucaccuccu

SEQ ID NO: 15 - RNA, 1492 nt, Staphylococcus caprae
agaguuugauccuggcucaggaugaacgcuggcggcgugccuaauacaugcaagucgagcgaacagacgaggagcuugcuccucugacguuagcggcggacgggugaguaacacguggauaaccuaccuauaagacugggauaacuucgggaaaccggagcuaauaccggauaacauguugaaccgcaugguucaacagugaaagacggucuugcugucacuuauagauggauccgcgccgcauuagcuaguugguaagguaacggcuuaccaaggcaacgaugcguagccgaccugagagggugaucggccacacuggaacugagacacgguccagacuccuacgggaggcagcaguagggaaucuuccgcaaugggcgaaagccugacggagcaacgccgcgugagugaagaaggucuucggaucguaaaacucuguuauuagggaagaacaaauguguaaguaacuaugcacgucuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccucugaccccucuagagauagaguuuuccccuucgggggacagagugacagguggugcaugguugucgucagcucgugucgugagauguuggguuaagucccgcaacgagcgcaacccuuaagcuuaguugccaucauuaaguugggcacucuaaguugacugccggugacaaaccggaggaagguggggaugacgucaaaucaucaugccccuuaugauuugggcuacacacgugcuacaauggacaauacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccuuuuggagcuagccgucgaaggugggacaaaugauuggggu

SEQ ID NO: 16 - RNA, 1494 nt, Staphylococcus arlettae
agaguuugauccuggcucaggaugaacgcuggcggcgugccuaauacaugcaagucgagcgaacagauaaggagcuugcuccuuugacguuagcggcggacgggugaguaacacguggguaaccuaccuauaagacuggaauaacuccgggaaaccggggcuaaugccggauaacauuuagaaccgcaugguucuaaagugaaagaugguuuugcuaucacuuauagauggacccgcgccguauuagcuaguugguaagguaauggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggguuucggcucguaaaacucuguuauuagggaagaacaaacguguaaguaacugugcacgucuugacgguaccuaaucagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggagacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaccacucuagagauagaguuuuccccuucgggggacaaagugacagguggugcaugguugucgucagcucgugucgugagauguuggguuaagucccgcaacgagcgcaacccuuaaacuuaguugccagcauuuaguugggcacucuagguugacugccggugacaaaccggaggaagguggggaugacgucaaaucaucaugccccuuaugauuugggcuacacacgugcuacaauggacaauacaaagggcagcuaaaccgcgaggucaugcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauuggggu

SEQ ID NO: 17 - RNA, 1494 nt, Staphylococcus kloosii
agaguuugauccuggcucaggaugaacgcuggcggcgugccuaauacaugcaagucgagcgaacagauaaggagcuugcuccuuugacguuagcggcggacgggugaguaacacguggguaaccuaccuauaagacuggaauaacuucgggaaaccgaagcuaaugccggauaacauguagaaccgcaugguucuacagugaaagauggccuugcuaucacuuauagauggacccgcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaagcucuguuauuagggaagaacaagugcguaaguaacugugcgcaccuugacgguaccuaaucagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggagacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaccacucuagagacagagcuuuccccuucgggggacaaagugacagguggugcaugguugucgucagcucgugucgugagauguuggguuaagucccgcaacgagcgcaacccuuaagcuuaguugccaucauuaaguugggcacucuaaguugacugccggugacaaaccggaggaagguggggaugacgucaaaucaucaugccccuuaugauuugggcuacacacgugcuacaauggacaauacaaagggcagcuaaaccgcgaggucaugcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauuggggu

SEQ ID NO: 18 - RNA, 1493 nt, Staphylococcus carnosus
agaguuugauccuggcucaggaugaacgcuggcggcgugccuaauacaugcaagucgagcgaacagaugaggagcuugcuccucugauguuagcggcggacgggugaguaacacguggguaaccuaccuauaagacuggaauaacuccgggaaaccggggcuaaugccggauaauaugcagaaccgcaugguucugcaaugaaagacgguuuugcugucacuuauagauggacccgcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuauuagggaagaacaagugcguagguaacuaugcgcaccuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucugcaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacggccgcaaggcugaaacucaaaggaauugacggggacccgcacaagcgguggagcaugugguuuaauucgaagcaacgcgaagaaccuuaccaaaucuugacauccuuugaccgcucuagagauagagucuuccccuucgggggacaaagugacagguggugcaugguugucgucagcucgugucgugagauguuggguuaagucccgcaacgagcgcaacccuuaagcuuaguugccagcauuaaguugggcacucuaaguugacugccggugacaaaccggaggaagguggggaugacgucaaaucaucaugccccuuaugauuugggcuacacacgugcuacaauggacaguacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccuuuuaggagcuagccgucgaaggugggacagaugauuggggu

SEQ ID NO: 19 - RNA, 1497 nt, Staphylococcus vitulinus
agaguuugauccuggcucaggacgaacgcuggcggcgugccuaauacaugcaagucgagcgaacagaugagaagcuugcuucucugauguuagcggcggacgggugaguaacacguggguaaccuaccuauaagacugggauaacuccgggaaaccggggcuaauaccugauaauauuuugaaccgcaugguucaauagugaaagacggcuucggcugucacuuauagauggacccgcgccguauuagcuaguugguaagguaauggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuuaggaucguaaaacucuguuguuagggaagaacaaauuuguuaguaacugaacaagucuugacgguaccuaaccagaaagccacggcuaacuacgugccagcagccgcgguaauacguagguggcaagcguuauccggaauuauugggcguaaagcgcgcguaggcgguuucuuaagucugaugugaaaguccacggcucaaccguggaaggucauuggaaacuggggaacuugagugcagaagaggagaguggaauuccauguguagcggugaaaugcgcagagauauggaggaacaccaguggcgaaggcggcucucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaucgcucuagagauagaguuuuccccuucgggggacaaagugacagguggugcaugguugucgucagcucgugucgugagauguuggguuaagucccgcaacgagcgcaacccuuaagcuuaguugccaucauuaaguugggcacucuagguugacugccggugacaaaccggaggaagguggggaugacgucaaaucaucaugccccuuaugauuugggcuacacacgugcuacaauggauaauacaaagggcagcgaaaccgcgaggucaagcaaaucccauaaaauuauucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccuuuuauuaggagcuagccgucgaaggugggacagaugauuggggu

SEQ ID NO: 20 - RNA, 1475 nt, Staphylococcus xylosus
gaugaacgcuggcggcgugccuaauacaugcaagucgagcgaacagauaaggagcuugcuccuuugaaguuagcggcggacgggugaguaacacguggguaaccuaccuauaagacugggauaacuucgggaaaccggagcuaauaccggauaacauuuagaaccgcaugguucuaaagugaaagaugguuuugcuaucacuuauagauggacccgcgccguauuagcuaguugguaagguaacggcuuaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaaggguuucggcucguaaaacucuguuauuagggaagaacaaauguguaaguaacugugcacaucuugacgguaccuaaucagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaaaacucuagagauagagccuuccccuucgggggacaaagugacagguggugcaugguugucgucagcucgugucgugagauguuggguuaagucccgcaacgagcgcaacccuuaagcuuaguugccaucauuaaguugggcacucuagguugacugccggugacaaaccggaggaagguggggaugacgucaaaucaucaugccccuuaugauuugggcuacacacgugcuacaauggacaauacaaagggcagcuaaaccgcgaggucaugcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauuggggug

SEQ ID NO: 21 - RNA, 1080 nt, Unknown
  Staphylococcus argensis
uuuugguuuuggagcgcgcuaacugcagucgagcgacagacggggagcuugcucccccgacguuagcggcggacgggugaguaacacguggguaaccuaccuauaagacuggaauaacuccgggaaaccggagcuaaugccggauaacauguuggaccgcaugguccuacagugaaagacggucuugcugucacuuauagauggacccgcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggccuucgggucguaaaacucuguuauuagggaagaacaaauguguaaguaacuaugcacgucuugacgguaccuaaucagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggaaacuugagugcaggagaggaaaguggaauuccauguguagcggugaaaugcgcagagauauggaggaacaccaguggcgaaggcgacuuucuggucugcaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcggugaagcaugugguuuaauucgaagcaaccccgaagaaccuuaccaaaucuuugacauccuuuugcccccucuagagauaaaaguuucccccuuccgggggacaaaauugacaggugggugcauuguuuguccuucaccuccuuguccuugagaauguuuggguu

SEQ ID NO: 22 - RNA, 1288 nt, Staphylococcus capitis
gcuugcuccucugacguuagcggcggacgggugaguaacacguggauaaccuaccuauaagacugggauaacuucgggaaaccggagcuaauaccggauaacauguugaaccgcaugguucaacagugaaagacggucuugcugucacuuauagauggauccgcgccgcauuagcuaguugguaagguaacggcuuaccaaggcaacgaugcguagccgaccugagagggugaucggccacacuggaacugagacacgguccagacuccuacgggaggcagcaguagggaaucuuccgcaaugggcgaaagccugacggagcaacgccgcgugagugaagaaggucuucggaucguaaaacucuguuauuagggaagaacaaauguguaaguaacuaugcacgucuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccucugaucccucuagagauagaguuuuccccuucgggggacagagugacagguggugcaugguugucgucagcucgugucgugagauguuggguuaagucccgcaacgagcgcaacccuuaagcuuaguugccaucauuaaguugggcacucuaaguugacugccggugacaaaccggaggaagguggggaugacgucaaaucaucaugccccuuaugauuugggcuacacacgugcuacaauggacaauacaaaggguagcgaaaccgcgaggucaagcaaaucccauaaaguuguucucaguucggauuguagucugcaacucgacuacaugaagcuggaaucgcuaguaaucguagauc

SEQ ID NO: 23 - RNA, 1537 nt, Unknown
  Staphylococcus devriesei
uagaguuugauccuggcucaggaugaacgcuggcggcgugccuaauacaugcaagucgagcgaacagauaaggagcuugcuccuuugacguuagcggcggacgggugaguaacacguggguaaccuaccuauaagacugggauaacuucgggaaaccggagcuaauaccggauaauauuucgaaccgcaugguucgauagugaaagaugguuuugcuaucacuuauagauggauccgcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuauuagggaagaacauacguguaaguaacuaugcacgucuugacgguaccuaaucagaaagccacggcuaacuacgugccagcagccgcgguaauacguagguggcaagcguuauccggaauuauugggcguaaagcgcgcguaggcgguuuuuuaagucugaugugaaaucccacggcucaaccguggagggucauuggaaacuggaaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaccgcucuagagauagaguuuuccccuucgggggacaaagugacagguggugcaugguugucgucagcucgugucgugagauguuggguuaagucccgcaacgagcgcaacccuuaaacuuaguugccagcauucaguugggcacucuaaguugacugccggugacaaaccggaggaagguggggaugacgucaaaucaucaugccccuuaugauuugggcuacacacgugcuacaauggacaauacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuuggagcuagccgucgaaggugggacaaaugauuggggugaagucguaacaagguagccguaucggaaggugcggcuggauc

SEQ ID NO: 24 - RNA, 1528 nt, Staphylococcus sciuri
gaugaacgcuggcggcgugccuaauacaugcaagucgagcgaacagaugagaagcuugcuucucugauguuagcggcggacgggugaguaacacguggguaaccuaccuauaagacugggauaacuccgggaaaccggggcuaauaccggauaauauuuugaaccgcaugguucaauagugaaagacgguuucggcugucacuuauagauggacccgcgccguauuagcuaguugguaagguaacggcuuaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuguuagggaagaacaaauuuguuaguaacugaacaagucuugacgguaccuaaccagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggaaacuugagugcagaagaggagaguggaauuccauguguagcggugaaaugcgcagagauauggaggaacaccaguggcgaaggcggcucucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaccgcucuagagauagagucuuccccuucgggggacaaagugacagguggugcaugguugucgucagcucgugucgugagauguuggguuaagucccgcaacgagcgcaacccuuaagcuuaguugccaucauuaaguugggcacucuagguugacugccggugacaaaccggaggaagguggggaugacgucaaaucaucaugccccuuaugauuugggcuacacacgugcuacaauggauaauacaaagggcagcgaauccgcgaggccaagcaaaucccauaaaauuauucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccuuuuaggagcuagccgucgaaggugggacaaaugauuggggugaagucguaacaagguagccguaucggaaggugcggcuggaucaccuccuuucu

SEQ ID NO: 25 - RNA, 1544 nt, Staphylococcus hominis
  misc_feature (11)..(12): n is a, c, g, or u
agaguuugaunnuggcucaggaugaacgcuggcggcgugccuaauacaugcaagucgagcgaacagacgaggagcuugcuccuuugacguuagcggcggacgggugaguaacacguggguaaccuaccuauaagacugggauaacuucgggaaaccggagcuaauaccggauaauauuucgaaccgcaugguucgauagugaaagauggcuuugcuaucacuuauagauggaccugcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuauuagggaagaacaaacguguaaguaacugugcacgucuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucucuaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugacccuucuagagauagaaguuuccuucgggggacaaagugacagguggugcaugguugucgucagcucgugucgugagauguuggguuaagucccgcaacgagcgcaacccuuaagcuuaguugccaucauuaaguugggcacucuaaguugacugccggugacaaaccggaggaagguggggaugacgucaaaucaucaugccccuuaugauuugggcuacacacgugcuacaauggacaauacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuggagcuagccgucgaaggugggacaaaugauuggggugaagucguaacaagguagccguaucggaaggugcggcuggaucaccuccuuucu

SEQ ID NO: 26 - RNA, 1477 nt, Unknown
  Staphylococcus microti
ggaugaacgcuggcggcgugccuaauacaugcaagucgagcgaacagacgaggugcuugcaccucugacguuagcggcggacgggugaguaacacguggguaaccuaccuauaagacugggauaacuccgggaaaccggggcuaauaccggauaacauauugaaccgcaugguucaauagugaaagacgguuuugcugucacuuauagauggacccgcgccguauuagcuaguuggugagguaacggcucaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaagcucuguuauuagggaagaacaaacguguaaguaacugugcacgucuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcggcuuucuggucugcaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacggucgcaagacugaaacucaaaggaauugacggggacccgcacaagcgguggagcaugugguuuaauucgaagcaacgcgaagaaccuuaccaaaucuugacauccuuugaccgcacuagagauaguguuuuccucuucggaggacaaagugacagguggugcaugguugucgucagcucgugucgugagauguuggguuaagucccgcaacgagcgcaacccuugagcuuaguugccaucauuaaguugggcacucuaagcugacugccggugacaaaccggaggaagguggggaugacgucaaaucaucaugccccuuaugauuugggcuacacacgugcuacaauggacauuacaaagggcagcaaaaccgcgaggucaagcaaaucccauaaagauguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuggagcuagccgucgaaggugggacaaaugauuggggugaag

SEQ ID NO: 27 - RNA, 1493 nt, Unknown
  Staphylococcus piscifermentans
agaguuugauccuggcucaggaugaacgcuggcggcgugccuaauacaugcaagucgagcgaacagacgaggagcuugcuccucugacguuagcggcggacgggugaguaacacguggguaaccuaccuauaagacuggaauaacuccgggaaaccggggcuaaugccggauaauaugcggaaccgcaugguuccgcaaugaaagacgguuuugcugucacuuauagauggacccgcgccguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuauuagggaagaacaagugcguagguaacuaugcgcaccuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucugcaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacggccgcaaggcugaaacucaaaggaauugacggggacccgcacaagcgguggagcaugugguuuaauucgaagcaacgcgaagaaccuuaccaaaucuugacauccuuugaccgcucuagagauagagucuuccccuucgggggacaaagugacagguggugcaugguugucgucagcucgugucgugagauguuggguuaagucccgcaacgagcgcaacccuuaagcuuaguugccagcauuaaguugggcacucuaaguugacugccggugacaaaccggaggaagguggggaugacgucaaaucaucaugccccuuaugauuugggcuacacacgugcuacaauggacaauacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccuuuuaggagcuagccgucgaaggugggacagaugauuggggu

SEQ ID NO: 28 - RNA, 819 nt, Unknown
  Staphylococcus schweitzeri
gccugacggagcacgccgcgugagugaugaaggucuucggaucguaaaacucuguuauuagggaagaacauauguguaaguaacugugcacaucuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugacaacucuagagauagagcuuuccucuucgggggacaaagugacagguggugcaugguugucgucagcucgugucgugagauguuggguuaagucccgcaacgagcgcaacccuuaagcuuaguugccaucauuaaguugggcacucuaaguugacugccggugacaaaccggaggaagguggggaugacgucaaauca

SEQ ID NO: 29 - RNA, 1476 nt, Staphylococcus simulans
aggaugaacgcuggcggcgugccuaauacaugcaagucgagcgaacagacgaggagcuugcuccucugacguuagcggcggacgggugaguaacacguggguaaccuaccuauaagacugggauaacuccgggaaaccggggcuaauaccggauaacacaugaaaccgcaugguuucaugaugaaagacgguuuugcugucacuuauagauggacccgcggcguauuagcuaguugguaagguaacggcuuaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuucggaucguaaaacucuguuauuagggaagaacaaggauguaaguaacuaugcaucccuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucugcaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacggccgcaaggcugaaacucaaaggaauugacggggacccgcacaagcgguggagcaugugguuuaauucgaagcaacgcgaagaaccuuaccaaaucuugacauccuuugacaacucuagagauagagcuuuccccuucgggggacaaagugacagguggugcaugguugucgucagcucgugucgugagauguuggguuaagucccgcaacgagcgcaacccuuaagcuuaguugccagcauuaaguugggcacucuaaguugacugccggugacaaaccggaggaagguggggaugacgucaaaucaucaugccccuuaugauuugggcuacacacgugcuacaauggacgguacaaagggcagcgaacccgcgaggucaagcaaaucccauaaagccguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccuuuuaggaacuagccgucgaaggugggacaaaugauuggggug

SEQ ID NO: 30 - RNA, 1548 nt, Unknown
  Staphylococcus succinus
uggagaguuugauccuggcucaggaugaacgcuggcggcgugccuaauacaugcaagucgagcgaacggauaaggagcuugcuccuuugaaguuagcggcggacgggugaguaacacguggguaaccuaccuauaagacuggaauaacuucgggaaaccggagcuaaugccggauaacauauagaaccgcaugguucuauagugaaagaugguuuugcuaucacuuauagauggacccgcgccguauuagcuaguugguaagguaauggcuuaccaaggcgacgauacguagccgaccugagagggugaucggccacacuggaacugagacacgguccagacuccuacgggaggcagcaguagggaaucuuccgcaaugggcgaaagccugacggagcaacgccgcgugagugaugaagguuuucggaucguaaaacucuguuauuagggaagaacaaaugcguaaguaacugugcgcaucuugacgguaccuaaucagaaagccacggcuaacuacgugccagcagccgcgguaauacguagguggcaagcguuauccggaauuauugggcguaaagcgcgcguaggcgguuucuuaagucugaugugaaagcccacggcucaaccguggagggucauuggaaacugggaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaaaacucuagagauagagccuuccccuucgggggacaaagugacagguggugcaugguugucgucagcucgugucgugagauguuggguuaagucccgcaacgagcgcaacccuuaagcuuaguugccaucauuaaguugggcacucuagguugacugccggugacaaaccggaggaagguggggaugacgucaaaucaucaugccccuuaugauuugggcuacacacgugcuacaauggacaauacaaagggcagcuaaaccgcgaggucaugcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuauggagcuagccgucgaaggugggacaaaugauuggggugaagucguaacaagguagccguaucggaaggugcggcuggaucaccuccuu

SEQ ID NO: 31 - RNA, 1492 nt, Staphylococcus lugdunensis
agaguuugauccuggcucaggaugaacgcuggcggcgugccuaauacaugcaagucgagcgaacagauaaggagcuugcuccuuugacguuagcggcggacgggugaguaacacguggguaaccuaccuauaagacugggacaacuucgggaaaccggagcuaauaccggauaauauguugaaccgcaugguucaauagugaaagaugguuuugcuaucacuuauagauggacccgcgccguauuagcuaguuggugagguaacggcucaccaaggcaacgauacguagccgaccugagagggugaucggccacacuggaacugagacacgguccagacuccuacgggaggcagcaguagggaaucuuccgcaaugggcgaaagccugacggagcaacgccgcgugagugaugaaggucuuaggaucguaaaacucuguuauuagggaagaacaaacguguaaguaacugugcacgucuugacgguaccuaaucagaaagccacggcuaacuacgugccagcagccgcgguaauacguagguggcaagcguuauccggaauuauugggcguaaagcgcgcguaggcgguuuuuuaagucugaugugaaagcccacggcucaaccguggagggucauuggaaacuggaaaacuugagugcagaagaggaaaguggaauuccauguguagcggugaaaugcgcagagauauggaggaacaccaguggcgaaggcgacuuucuggucuguaacugacgcugaugugcgaaagcguggggaucaaacaggauuagauacccugguaguccacgccguaaacgaugagugcuaaguguuaggggguuuccgccccuuagugcugcagcuaacgcauuaagcacuccgccuggggaguacgaccgcaagguugaaacucaaaggaauugacggggacccgcacaagcgguggagcaugugguuuaauucgaagcaacgcgaagaaccuuaccaaaucuugacauccuuugaccgcucuagagauagagucuuccccuucgggggacaaagugacagguggugcaugguugucgucagcucgugucgugagauguuggguuaagucccgcaacgagcgcaacccuuaagcuuaguugccaucauuuaguugggcacucuaaguugacugccggugacaaaccggaggaagguggggaugacgucaaaucaucaugccccuuaugauuugggcuacacacgugcuacaauggacaauacaaagggcagcgaaaccgcgaggucaagcaaaucccauaaaguuguucucaguucggauuguagucugcaacucgacuacaugaagcuggaaucgcuaguaaucguagaucagcaugcuacggugaauacguucccgggucuuguacacaccgcccgucacaccacgagaguuuguaacacccgaagccgguggaguaaccauuuggagcuagccgucgaaggugggacaaaugauuggggu

(19) United States
(12) Patent Application Publication
(10) Pub. No.: US 2022/0170060 A1
(43) Pub. Date: Jun. 2, 2022

(54) MICROORGANISM BELONGING TO GENUS STAPHYLOCOCCUS PRODUCING ALLULOSE AND METHOD FOR PREPARING ALLULOSE USING THE SAME

(71) Applicant: CJ CHEILJEDANG CORPORATION, Seoul (KR)

(72) Inventors: Su Jin KIM, Seoul (KR); Hyun CHI, Seoul (KR); Eunsoo HONG, Seoul (KR); Yang Hee KIM, Seoul (KR); Taek Beom KIM, Seoul (KR); Junseok GWAK, Seoul (KR); Seong Bo KIM, Seoul (KR); Eun Jung CHOI, Seoul (KR)

(21) Appl. No.: 17/437,227

(22) PCT Filed: Mar. 4, 2020
(86) PCT No.: PCT/KR2020/003061

§ 371 Date: Sep. 8, 2021

(30) Foreign Application Priority Data
Mar. 8, 2019 (KR) 10-2019-0026633

(51) Int. Cl. C12P 19/02; C12N 1/20

(52) U.S. Cl. CPC C12P 19/02; C12N 1/20

(57) ABSTRACT

The present application relates to a microorganism that produces allulose and a method for preparing allulose using the same.

### TECHNICAL FIELD

The present application relates to a microorganism that produces allulose and a method for preparing allulose using the same.

### BACKGROUND ART

D-Allulose is a C3 epimer of D-fructose and is a monosaccharide known as a rare sugar that is present in a significantly small amount in nature. The sweetness of D-allulose is about 70% of the sweetness of sugar, but the caloric value thereof is almost zero. D-Allulose has functions of suppressing the elevation of blood sugar levels, lipid synthesis, and the like, and thus it is receiving much attention as a new food ingredient that can be used in functional foods.

Due to these properties, allulose is considered for use in various foods as a sugar substitute sweetener. However, allulose is present in a significantly small amount in nature, and there is thus a growing need for a method by which allulose can be efficiently manufactured.

As the conventionally known production method of allulose, a chemical method has been known in which allulose is produced from D-fructose by using the catalytic action of molybdate ions or by heating D-fructose with ethanol and triethylamine. Such a chemical method has disadvantages such as a low allulose production yield and high manufacturing cost.

In order to solve such problems, a biological method for producing allulose from fructose using an epimerase derived from microorganisms has been studied. Starting with a technology for producing allulose using genetically modified microorganisms with a D-allulose 3-epimerization gene derived from Agrobacterium tumefaciens, a method for producing allulose from D-fructose by separating microbial strains from the natural environment or food and using these microbial strains has also been recently reported (Korean Patent Publication No. 10-2011-0035805, Korean Patent Publication No. 10-1804778, Korean Patent Publication No. 10-2017-0067070, and Korean Patent Publication No. 10-2016-00817220). Biological technologies for producing allulose have been developed, but there is an urgent need to discover new microbial resources that can produce allulose because of the low thermal stability of microorganisms used as biocatalysts or high production cost.

With this background, as a result of diligent studies to develop microorganisms that can produce allulose even in a high-temperature environment, it has been confirmed that microorganisms belonging to the genus Staphylococcus can produce allulose, and thus the present application has been completed.

### DISCLOSURE

### Technical Problem

The present application provides a composition for allulose production comprising a microorganism belonging to the genus Staphylococcus.

The present application provides a method for preparing allulose by using the composition.

### Technical Solution

The technical solution will be described in detail as follows. Meanwhile, each description and embodiment disclosed in the present application may also be applied to other descriptions and embodiments. That is, all combinations of various elements disclosed in the present application fall within the scope of the present application. Further, the scope of the present application is not limited by the specific description below.

An aspect of the present application may provide a composition for allulose production comprising a microorganism belonging to the genus Staphylococcus or a culture of the microorganism belonging to the genus Staphylococcus.

Specifically, the microorganism belonging to the genus Staphylococcus may be any one selected from the group consisting of Staphylococcus agnetis, Staphylococcus argensis, Staphylococcus argenteus, Staphylococcus adettae, Staphylococcus aureus, Staphylococcus aurculars, Staphylococcus capitis, Staphylococcus caprae, Staphylococcus camosus, Staphylococcus chromogenes, Staphylococcus cohnii, Staphylococcus condiment, Staphylococcus comubiensis, Staphylococcus delphini, Staphylococcus devriesei, Staphylococcus edaphicus, Staphylococcus epidermidis, Staphylococcus equorum, Staphylococcus faecalis, Staphylococcus felis, Staphylococcus fleurettii, Staphylococcus gallinarum, Staphylococcus haemolyticus, Staphylococcus hominis, Staphylococcus hyicus, Staphylococcus intermedius, Staphylococcus kloosii, Staphylococcus leei, Staphylococcus lentus, Staphylococcus lugdunensis, Staphylococcus lutrae, Staphylococcus lyticans, Staphylococcus massiliensis, Staphylococcus microti, Staphylococcus muscae, Staphylococcus nepalensis, Staphylococcus pasteuri, Staphylococcus petrasii, Staphylococcus pettenkoferi, Staphylococcus piscifermentans, Staphylococcus pseudintermedius, Staphylococcus pseudolugdunensis, Staphylococcus rostri, Staphylococcus saccharolyticus, Staphylococcus saprophyticus, Staphylococcus schleifer, Staphylococcus schweitzeri, Staphylococcus sciuri, Staphylococcus simiae, Staphylococcus simulans, Staphylococcus stepanovicii, Staphylococcus succinus, Staphylococcus vitulinus, Staphylococcus wamer, and Staphylococcus xylosus, but is not limited thereto.

The microorganism belonging to the genus Staphylococcus may be more specifically any one selected from the group consisting of Staphylococcus camosus, Staphylococcus xylosus, Staphylococcus vitulinus, Staphylococcus epidermidis, Staphylococcus warner, Staphylococcus haemolyticus, Staphylococcus intermedius, Staphylococcus saprophyticus, Staphylococcus cohnii, Staphylococcus muscae, Staphylococcus lentus, Staphylococcus chromogenes, Staphylococcus caprae, Staphylococcus auricularis, Staphylococcus gallinarum, Staphylococcus arlettae, Staphylococcus equorum, Staphylococcus kloosii, Staphylococcus delphini, and Staphylococcus pasteuri, still more specifically Staphylococcus camosus, Staphylococcus xylosus, or Staphylococcus vitulinus, but is not limited thereto.

In the present application, D-allulose (hereinafter referred to as allulose) is an epimer of D-fructose and is prepared from fructose by an epimerase. In the present application, D-allulose may be used interchangeably with psicose.

In the present application, the fructose used as a substrate may be obtained from sugar decomposed by a converting enzyme or from high-fructose corn syrup, or may be purchased as a commercial product, but is not limited thereto.

The microorganism belonging to the genus Staphylococcus of the present application has the activity of converting D-fructose to D-allulose. The microorganism belonging to the genus Staphylococcus produces D-allulose from D-fructose by using its metabolic system. Such a conversion reaction may be conducted within the microorganism or outside of the microorganism by way of the substances secreted, but a microorganism belonging to the genus Staphylococcus falls within the scope of the present application without limitation of the allulose producing process as long as it is a microorganism belonging to the genus Staphylococcus that can produce allulose from fructose.

Meanwhile, the microorganism belonging to the genus Staphylococcus of the present application may mean not only wild-type microorganisms but also mutant microorganisms including mutations that occur naturally or non-naturally. Specifically, mutations occurring non-naturally may be to mutate wild-type microorganisms or naturally mutated microorganisms using UV irradiation, radiation (gamma ray, X-ray), or chemical mutagens. A microorganism belonging to the genus Staphylococcus falls within the scope of the present application as long as it has the properties of the microorganism belonging to the genus Staphylococcus that has the ability to produce allulose from fructose even though the microorganism has a genetic trait different from that of a wild-type microorganism belonging to the genus Staphylococcus.

The microorganism belonging to the genus Staphylococcus of the present application exhibits heat resistance so as to be able to produce allulose even in a high-temperature environment, for example, at a temperature of 50° C. or more, and thus has an advantage of increasing the yield of allulose production.

The microorganism belonging to the genus Staphylococcus of the present application exhibits the activity of converting fructose to allulose, and a microorganism belonging to the genus Staphylococcus may fall within the scope of the present application as long as it has an allulose conversion rate that can be industrially used.

In the present application, the conversion rate may be expressed as the concentration of allulose produced by 12 hours of reaction/initial fructose concentration (1 wt %), and the microorganism belonging to the genus Staphylococcus of the present application may have a conversion rate of 0.1% or more, specifically 0.3% or more, 0.5% or more, 0.9% or more, 1.6% or more, 2.3% or more, 3.4% or more, 5.3% or more, 10.4% or more, or 24.5% or more, but the conversion rate is not limited thereto.

In the present application, the conversion rate may be measured by way of a method known in the art, and the method is not limited to a specific method. As an example, in the present application, the result of conversion reaction to allulose conducted at a pH of 7.5 and 55° C. for 12 hours was measured as the conversion rate, but the reaction conditions (for example, pH, temperature, time, and the like) may be appropriately selected by those skilled in the art to measure the conversion rate.

In particular, the microorganism belonging to the genus Staphylococcus is known not to have pathogenicity and thus has an advantage of being able to be used in various foods.

Accordingly, the microorganism belonging to the genus Staphylococcus of the present application may be nonpathogenic, but is not limited thereto. Specifically, the microorganism belonging to the genus Staphylococcus that is known as a nonpathogenic microorganism may be Staphylococcus argensis, Staphylococcus capitis, Staphylococcus devresei, Staphylococcus faecalis, Staphylococcus sciuri, Staphylococcus hominis, Staphylococcus lugdunensis, Staphylococcus microti, Staphylococcus piscifermentans, Staphylococcus schweitzeri, Staphylococcus simulans, Staphylococcus succinus, Staphylococcus arlettae, Staphylococcus auricularis, Staphylococcus caprae, Staphylococcus carnosus, Staphylococcus chromogenes, Staphylococcus cohnii, Staphylococcus delphini, Staphylococcus epidermidis, Staphylococcus equorum, Staphylococcus gallinarum, Staphylococcus haemolyticus, Staphylococcus intermedius, Staphylococcus kloosii, Staphylococcus lentus, Staphylococcus muscae, Staphylococcus pasteuri, Staphylococcus saprophyticus, Staphylococcus vitulinus, Staphylococcus warneri, or Staphylococcus xylosus, but is not limited thereto.

In the present application, nonpathogenic microorganisms mean microorganisms that do not bring symptoms of disease in individuals including humans, and mean safe strains corresponding to the internationally accepted biosafety level 1. Specifically, the nonpathogenic microorganisms mean all strains except Staphylococcus agnetis, Staphylococcus argenteus, Staphylococcus aureus, Staphylococcus cornubiensis, Staphylococcus felis, Staphylococcus fleurettii, Staphylococcus hyicus, Staphylococcus lutrae, Staphylococcus massiliensis, Staphylococcus nepalensis, Staphylococcus petrasii, Staphylococcus pettenkoferi, Staphylococcus pseudintermedius, Staphylococcus rostri, Staphylococcus schleifer, Staphylococcus simiae, and Staphylococcus stepanovicii strains that correspond to the biosafety level 2 or more among the microorganisms belonging to the genus Staphylococcus.

The nonpathogenic microorganism belonging to the genus Staphylococcus of the present application does not adversely affect individuals including humans while having the ability to produce allulose.

In the present application, it has been confirmed that various microorganisms belonging to the genus Staphylococcus, in particular, nonpathogenic microorganisms, not only have the activity of converting fructose to allulose but also have genetic connectivity therebetween through the analysis of 16S rRNA of the microorganisms.

In the present application, “16S rRNA” refers to 16S ribosomal RNA, the rRNA component of the 30S subunit of a prokaryotic ribosome, having a length of about 1,500 nucleotides. The 16S rRNA sequence is known to be a sequence generally used to identify prokaryotes since the 16S rRNA sequence is most highly conserved while exhibiting high base sequence diversity in some sections and, in particular, diversity rarely appears in the same species, whereas diversity appears between different species. In other words, the genetic connectivity may be determined by comparing the homology of the 16S rRNA sequence. It may be understood that prokaryotes have more similar genetic traits as the homology (similarity) of 16S rRNA sequence becomes higher. In the present application, the term “homology” or “identity” means the degree to which two given base sequences are related to each other, and may be expressed as a percentage.

The terms homology and identity may often be used interchangeably.

Conserved polynucleotide sequence homology or identity is determined by standard sequence alignment algorithms, and the default gap penalty established by the program being used may be used together. Substantially, homologous or identical sequences are capable of hybridizing generally to the full sequence or to at least about 50%, 60%, 70%, 80%, or 90% or more of the full length in moderate or highly stringent conditions. In hybridization, polynucleotides containing degenerate codons instead of codons in the polynucleotide are also contemplated.

Whether any two polynucleotide sequences have homology, similarity, or identity may be determined, for example, using default parameters as in Pearson et al. (1988) Proc. Natl. Acad. Sci. USA 85:2444 and known computer algorithms such as the “FASTA” program. Alternatively, whether any two polynucleotide sequences have homology, similarity, or identity may be determined using the Needleman-Wunsch algorithm (Needleman and Wunsch, 1970, J. Mol. Biol. 48:443-453) as performed in the Needleman program of the EMBOSS package (EMBOSS: The European Molecular Biology Open Software Suite, Rice et al., 2000, Trends Genet. 16:276-277) (version 5.0.0 or later). The GCG program package (Devereux, J. et al, Nucleic Acids Research 12:387 (1984)), BLASTP, BLASTN, FASTA (Atschul, S. F. et al., J MOLEC BIOL 215:403 (1990); Guide to Huge Computers, Martin J. Bishop, ed., Academic Press, San Diego, 1994; and CARILLO et al. (1988) SIAM J Applied Math 48:1073 are also included. For example, BLAST of the National Center for Biotechnology Information, the Clustal omega program (https://www.ebi.ac.uk/Tools/msa/clustalo), or ClustalW may be used to determine homology, similarity, or identity.

The homology, similarity or identity of polynucleotides may be determined by comparing the sequence information thereof, for example, using the GAP computer program such as Needleman et al. (1970), J Mol Biol. 48:443, for example, as known in Smith and Waterman, Adv. Appl. Math (1981) 2:482. In short, the homology, similarity, or identity of polynucleotides is defined as the value obtained by dividing the number of similarly aligned symbols (namely, nucleotides or amino acids) by the total number of symbols in the shorter of the two sequences by the GAP program. The default parameters for the GAP program may include (1) a unary numeral system comparison matrix (containing a value of 1 for identity and 0 for non-identity) and a weighted comparison matrix of Gribskov et al., (1986) Nucl. Acids Res. 14:6745 (or EDNAFULL (EMBOSS version of NCBI NUC4.4) substitution matrix) as disclosed by Schwartz and Dayhoff, eds., Atlas Of Protein Sequence And Structure, National Biomedical Research Foundation, pp. 353-358 (1979); (2) a penalty of 3.0 for each gap and an additional penalty of 0.10 for each symbol in each gap (or a gap opening penalty of 10, a gap extension penalty of 0.5); and (3) no penalty for an end gap. Hence, as used in the present application, the term “homology” or “identity” refers to relatedness between sequences.

The composition for allulose production of the present application may comprise one, two, or more microorganism(s) belonging to the genus Staphylococcus or a culture thereof, but is not limited thereto.

The composition for allulose production of the present application comprises a microorganism belonging to the genus Staphylococcus that exhibits the activity of converting fructose to allulose or a culture thereof and thus can produce allulose.

In the present application, “culturing” means to grow a microorganism under appropriate artificially controlled environmental conditions, and “culture” means a product obtained by culturing a microorganism and may contain a microorganism or all substances secreted from the microorganism.

In the present application, culturing of a microorganism belonging to the genus Staphylococcus may be performed by way of a method widely known in the art. Specifically, the culturing may be performed batchwise, continuously, or fed-batchwise in a batch, injection and batch, or repeated injection and batch process, but is not limited thereto.

The medium used for culturing must meet the requirements for a specific strain in an appropriate manner, and the medium conditions for culturing of microorganisms belonging to the genus Staphylococcus are known.

Specifically, the sugar source that may be used in the medium includes sugars and carbohydrates such as glucose, saccharose, lactose, fructose, maltose, starch, and cellulose, oils and fats such as soybean oil, sunflower oil, castor oil, and coconut oil, fatty acids such as palmitic acid, stearic acid, and linoleic acid, alcohols such as glycerol and ethanol, and organic acids such as acetic acid. These substances may be used individually or as a mixture, but the way to use the sugar sources is not limited thereto.

The carbon source that may be used may be raw sugar or glucose, molasses containing a large amount of raw sugar, and specifically purified glucose, but is not limited thereto, and other carbon sources may be used in various ways.

The nitrogen source that may be used includes peptone, yeast extract, broth, malt extract, corn steep liquor, soybean meal, and urea or inorganic compounds such as ammonium sulfate, ammonium chloride, ammonium phosphate, ammonium carbonate, and ammonium nitrate. The nitrogen sources may also be used individually or as a mixture, but the way to use the nitrogen sources is not limited thereto.

The phosphorus source that may be used may include potassium dihydrogen phosphate or dipotassium hydrogen phosphate or the corresponding salt containing sodium.

The culture medium may contain a metal salt such as magnesium sulfate or iron sulfate required for growth. In addition to the substances, essential growth substances such as amino acids and vitamins may be used. Precursors suitable for the culture medium may be used. The above-described raw materials may be added batchwise or continuously in a manner suitable for the culture during the culturing process.

The pH of the culture may be adjusted during the culturing of a microorganism using basic compounds such as sodium hydroxide, potassium hydroxide, and ammonia or acidic compounds such as phosphoric acid or sulfuric acid in an appropriate manner. Foaming may be suppressed using an antifoaming agent such as fatty acid polyglycol ester. Oxygen or an oxygen-containing gas (for example, air) may be injected into the culture to maintain the aerobic condition.

The composition for allulose production of the present application may further comprise fructose, which is a substrate and/or an enzyme involved in the allulose production, in addition to the microorganism belonging to the genus Staphylococcus or a culture of the microorganism, but is not limited thereto.

The composition for allulose production of the present application may further contain arbitrary suitable excipients that are commonly used in the composition for allulose production. Such excipients may include, for example, a preservative, a wetting agent, a dispersing agent, a suspending agent, a buffering agent, a stabilizing agent, and an isotonizing agent, but are not limited thereto.

The composition for allulose production of the present application may further contain a metal ion or metal salt. The composition for allulose production of the present application may exhibit the activity of converting fructose to allulose since the metal ion or metal salt is contained in the composition. The metal ion or metal salt may be required in the process of producing allulose from fructose by a microorganism belonging to the genus Staphylococcus, for example, it may be required by the action of an enzyme that mediates the conversion process, but is not limited thereto. As for the metal ion or metal salt contained in the composition for allulose production of the present application, a metal ion or metal salt known to those skilled in the art may be appropriately selected as long as the composition can exhibit the activity of converting fructose to allulose. In an embodiment, the metal ion may be a divalent cation, specifically ions of one or more metals selected from the group consisting of Ni, Mg, Ni, Co, Mn, Fe, and Zn. More specifically, the composition for allulose production of the present application may further contain a metal salt. Still more specifically, the metal salt may be one or more selected from the group consisting of NiSO₄, MgSO₄, MgCl₂, NiCl₂, CoSO₄, CoCl₂, MnC₁₂, MnSO₄, FeSO₄, and ZnSO₄.

Another aspect of the present application may provide the use of a microorganism belonging to the genus Staphylococcus or a culture of the microorganism belonging to the genus Staphylococcus for allulose production.

The “microorganism belonging to the genus Staphylococcus” and “culture of a microorganism belonging to the genus Staphylococcus” are as described above.

Still another aspect of the present application provides a method for preparing allulose, which includes bringing the composition into contact with fructose to convert the fructose to allulose.

Specifically, the microorganism belonging to the genus Staphylococcus may be any one selected from the group consisting of Staphylococcus agnetis, Staphylococcus argensis, Staphylococcus argenteus, Staphylococcus arettae, Staphylococcus aureus, Staphylococcus auricularis, Staphylococcus capitis, Staphylococcus caprae, Staphylococcus carnosus, Staphylococcus chromogenes, Staphylococcus cohnii, Staphylococcus condiment, Staphylococcus comubiensis, Staphylococcus delphini, Staphylococcus devriesei, Staphylococcus edaphicus, Staphylococcus epidermidis, Staphylococcus equorum, Staphylococcus faecalis, Staphylococcus felis, Staphylococcus fleurettii, Staphylococcus gallinarum, Staphylococcus haemolyticus, Staphylococcus hominis, Staphylococcus hyicus, Staphylococcus intermedius, Staphylococcus kloosii, Staphylococcus leei, Staphylococcus lentus, Staphylococcus lugdunensis, Staphylococcus lutrae, Staphylococcus lyticans, Staphylococcus massiliensis, Staphylococcus microti, Staphylococcus muscae, Staphylococcus nepalensis, Staphylococcus pasteuri, Staphylococcus petrasii, Staphylococcus pettenkoferi, Staphylococcus piscifermentans, Staphylococcus pseudintermedius, Staphylococcus pseudolugdunensis, Staphylococcus rostri, Staphylococcus saccharolyticus, Staphylococcus saprophyticus, Staphylococcus schleiferi, Staphylococcus schweitzeri, Staphylococcus sciuri, Staphylococcus simiae, Staphylococcus simulans, Staphylococcus stepanovicii, Staphylococcus succinus, Staphylococcus vitulinus, Staphylococcus warneri, and Staphylococcus xylosus, but is not limited thereto.

The microorganism belonging to the genus Staphylococcus may be more specifically any one selected from the group consisting of Staphylococcus carnosus, Staphylococcus xylosus, Staphylococcus vitulinus, Staphylococcus epidermidis, Staphylococcus warner, Staphylococcus haemolyticus, Staphylococcus intermedius, Staphylococcus saprophyticus, Staphylococcus cohnii, Staphylococcus muscae, Staphylococcus lentus, Staphylococcus chromogenes, Staphylococcus caprae, Staphylococcus auricularis, Staphylococcus gallinarum, Staphylococcus arlettae, Staphylococcus equorum, Staphylococcus kloosii, Staphylococcus delphini, and Staphylococcus pasteuri, still more specifically Staphylococcus camosus, Staphylococcus xylosus, or Staphylococcus vitulinus, but is not limited thereto.

The preparation method of the present application may additionally include obtaining fructose from sugar and glucose, but is not limited thereto. The method for obtaining fructose from sugar and glucose, in particular, a production method using an enzyme, is known in the art.

The preparation method of the present application has an advantage of discharging fewer pollutants and increasing the yield since fructose can be converted to allulose even in a high-temperature environment by this preparation method.

Specifically, the temperature in the converting fructose to allulose may be 40° C. to 70° C., specifically 50° C. to 70° C. The culturing may be continuously performed until a desired amount of allulose is produced, and the culturing time may be specifically 5 to 120 hours, more specifically 10 to 30 hours, but is not limited thereto. In the preparation method of the present application, conversion of fructose to allulose may be performed at a pH of 5.0 to 9.0, specifically a pH of 6.0 to 8.0.

The preparation method of the present application may further include recovering allulose converted by a microorganism belonging to the genus Staphylococcus, but is not limited thereto.

Specifically, allulose may be recovered by crushing the microorganism belonging to the genus Staphylococcus, or allulose may be separated from the culture of the microorganism belonging to the genus Staphylococcus, but the recovery method is not limited to a specific method as long as allulose converted by the microorganism belonging to the genus Staphylococcus can be recovered.

Separation of allulose may be performed by way of conventional methods known in the art. As such a separation method, methods such as centrifugation, filtration, ion-exchange chromatography, and crystallization may be used. For example, the culture may be centrifuged at a low speed to remove biomass, and the obtained supernatant may be subjected to ion-exchange chromatography for separation of allulose, but the separation method is not limited thereto.

The preparation method of the present application may further include purifying allulose, but is not limited thereto. The purification may be performed by way of a conventionally used method, and non-limiting examples thereof may include dialysis, precipitation, adsorption, electrophoresis, ion-exchange chromatography, and fractional crystallization. The purification may be performed by way of only one method or the combination of two or more methods. For example, the reaction mixture having produced allulose may be purified through chromatography, and the separation of sugar by chromatography may be performed using a difference in weak binding strength between the sugar to be separated and the metal ion attached to the ion-exchange resin.

The preparation method of the present application may further include performing decolorization or desalting or both decolorization and desalting before or after the allulose purifying step of the present application. By performing the decolorization and/or desalting, a more purified reaction mixture containing allulose without impurities may be obtained.

### Advantageous Effects

The microorganism belonging to the genus Staphylococcus of the present application has an effect of producing allulose even in a high-temperature environment.

### BRIEF DESCRIPTION OF DRAWINGS

FIG. 1 is a diagram for confirming the production of allulose by Staphylococcus delphini KCTC3592:

FIG. 2 is a diagram illustrating the phylogenetic tree of nonpathogenic Staphylococcus strains; and

FIG. 3 is a diagram illustrating the phylogenetic tree of 20 nonpathogenic Staphylococcus strains.

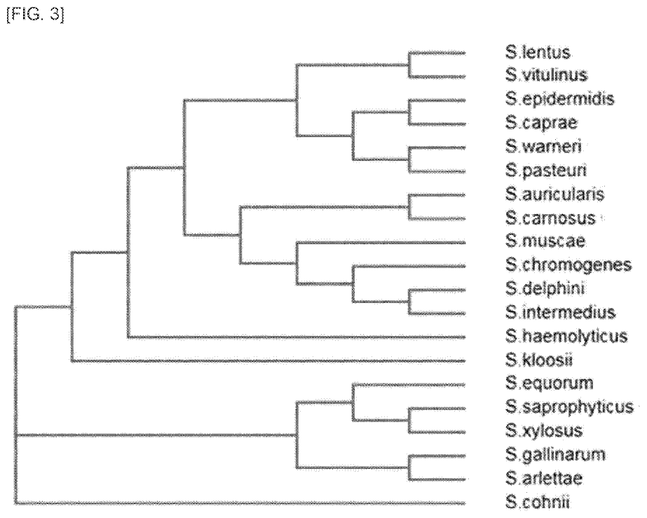

### DETAILED DESCRIPTION OF THE INVENTION

The best mode for carrying out the invention will be described in detail as follows. Meanwhile, each description and embodiment disclosed in the present application may also be applied to other descriptions and embodiments. That is, all combinations of various elements disclosed in the present application fall within the scope of the present application. Further, the scope of the present application is not limited by the specific description below.

In order to achieve the above object, the present application provides a composition for allulose production comprising a microorganism belonging to the genus Staphylococcus or a culture of the microorganism belonging to the genus Staphylococcus.

### EXAMPLES

Hereinafter, the present application will be described in more detail with reference to Examples. However, these Examples are for illustrative purposes only, and the scope of the present application is not limited to these Examples.

### Example 1: Confirmation of Allulose Production by Microorganism Belonging to Genus Staphylococcus

In order to confirm the ability of microorganisms belonging to the genus Staphylococcus to produce allulose, 32 nonpathogenic microorganisms belonging to the genus Staphylococcus were selected, and it was confirmed whether 20 microorganisms belonging to the genus Staphylococcus among these produced allulose from D-fructose.

Specifically, 2 species of Staphylococcus carnosus (KCTC3580, KACC13250), 2 species of Staphylococcus xylosus (KCTC3342, KACC16180), 4 species of Staphylococcus vitulinus (KACC15803, KACC15804, KACC15805, KACC13211), Staphylococcus delphini (KCTC3592), Staphylococcus equorum (KCTC3589), Staphylococcus epidermidis (KCTC1917), Staphylococcus cohnii (KCTC3574), Staphylococcus chromogenes (KCTC3579), Staphylococcus caprae (KCTC3583), Staphylococcus warneri (KCTC3340), Staphylococcus lentus (KCTC3577), Staphylococcus muscae (KCTC3576), Staphylococcus saprophyticus (KCTC3345), Staphylococcus pasteuri (KCTC13167), Staphylococcus intermedius (KCTC3344), Staphylococcus arlettae (KCTC3588), Staphylococcus kloosii (KCTC3590), Staphylococcus haemolyticus (KCTC3341), Staphylococcus gallinarum (KCTC3585), and Staphylococcus auricularis (KCTC3584) were distributed from the Korean Collection for Type Cultures (KTCT) and the Korean Agricultural Culture Collection (KACC).

Each of the microorganisms was inoculated into Tryptic Soy Broth (peptone 17 g/L, soytone 3 g/L, psicose 10 g/L, NaCl 5 g/L, K₂HPO₄ 2.5 g/L, agar 15 g/L) to which allulose was added at 1%, and cultured at 30° C. or 37° C. for 18 hours. Thereafter, the cultured microorganisms were recovered, washed with 0.85% (w/v) NaCl, and then used to perform the whole-cell conversion reaction.

To the microorganism at a concentration of 20% (w/w), 50 mM potassium phosphate buffer (pH 7.5) to which D-fructose was added at 1% (w/w) was added and suspended, and the suspension was subjected to the conversion reaction at 55° C. for 12 hours.

The supernatant of the conversion reaction product was subjected to HPLC analysis to confirm the allulose production. HPLC analysis was performed at a temperature of 80° C. and a flow velocity of 0.6 mL/min using water as a mobile phase solvent and a Refractive Index Detector (Agilent 1260 RID) of HPLC (Agilent, USA) equipped with Aminex HPX-87C column (BIO-RAD). The allulose conversion rate was calculated as the ratio of the weight of allulose produced after the reaction to the weight of substrate (D fructose) before the reaction (allulose concentration (12 h reaction)/initial fructose concentration (1 wt %)).

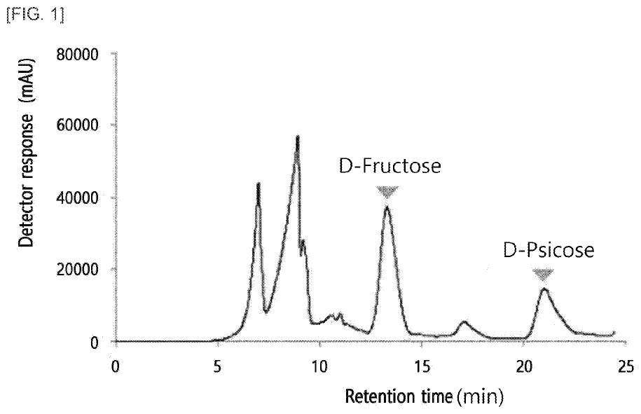

As a result, it has been confirmed that the 20 microorganisms belonging to the genus Staphylococcus all produce allulose from D-fructose (Table and FIG. 1).

TABLE 1

| Name of strain | Conversion rate (%) |
|---|---|
| Staphylococcus delphini KCTC3592 | 29.8 |
| Staphylococcus carnosus KCTC3580 | 26.2 |
| Staphylococcus carnosus KACC13250 | 25.7 |
| Staphylococcus equorum KCTC3589 | 24.5 |
| Staphylococcus epidermidis KCTC1917 | 15.8 |
| Staphylococcus cohnii KCTC3574 | 10.4 |
| Staphylococcus xylosus KCTC3342 | 8.0 |
| Staphylococcus xylosus KACC16180 | 8.0 |
| Staphylococcus chromogenes KCTC3579 | 7.8 |
| Staphylococcus caprae KCTC3583 | 5.3 |
| Staphylococcus vitulinus KACC15803 | 3.3 |
| Staphylococcus vitulinus KACC15804 | 3.4 |
| Staphylococcus warneri KCTC3340 | 2.7 |
| Staphylococcus lentus KCTC3577 | 2.9 |
| Staphylococcus vitulinus KACC15805 | 2.6 |
| Staphylococcus muscae KCTC3576 | 2.3 |
| Staphylococcus pasteuri KCTC13167 | 1.8 |
| Staphylococcus intermedius KCTC3344 | 1.7 |
| Staphylococcus saprophyticus KCTC3345 | 1.6 |
| Staphylococcus arlettae KCTC3588 | 1.2 |
| Staphylococcus vitulinus KACC13211 | 0.9 |
| Staphylococcus kloosii KCTC3590 | 0.8 |
| Staphylococcus haemolyticus KCTC3341 | 0.5 |
| Staphylococcus gallinarum KCTC3585 | 0.4 |
| Staphylococcus auricularis KCTC3584 | 0.3 |

### Example 2: Confirmation of 16S rRNA Similarity of Microorganisms Belonging to Genus Staphylococcus

### Example 2-1: Confirmation of 16S rRNA Similarity of Nonpathogenic Microorganisms Belonging to Genus Staphylococcus

Microorganisms classified as nonpathogenic (biosafety level 1) microorganisms belonging to the genus Staphylococcus in the present Example are as follows: Staphylococcus argensis, Staphylococcus capitis, Staphylococcus devriesei, Staphylococcus faecalis, Staphylococcus sciuri, Staphylococcus hominis, Staphylococcus lugdunensis, Staphylococcus microti, Staphylococcus piscifermentans, Staphylococcus schweitzeri, Staphylococcus simulans, Staphylococcus succinus, Staphylococcus arlettae, Staphylococcus auricularis, Staphylococcus caprae, Staphylococcus camosus, Staphylococcus chromogenes, Staphylococcus cohnii, Staphylococcus delphini, Staphylococcus epidermidis, Staphylococcus equorum, Staphylococcus gallinarum, Staphylococcus haemolyticus, Staphylococcus intermedius, Staphylococcus kloosii, Staphylococcus lentus, Staphylococcus muscae, Staphylococcus pasteuri, Staphylococcus saprophyticus, Staphylococcus vitulinus, Staphylococcus warneri, and Staphylococcus xylosus.

The sequence homology between strains was analyzed using the Clustal omega program (https://www.ebi.ac.uk/Tools/msa/clustalo) based on the 16s rRNA sequences of 31 microorganisms among the nonpathogenic microorganisms belonging to the genus Staphylococcus except Staphylococcus faecalis, of which the 16s rRNA sequence was not identified. The representative 16s rRNA sequences of the respective strains were confirmed from the NCBI database.

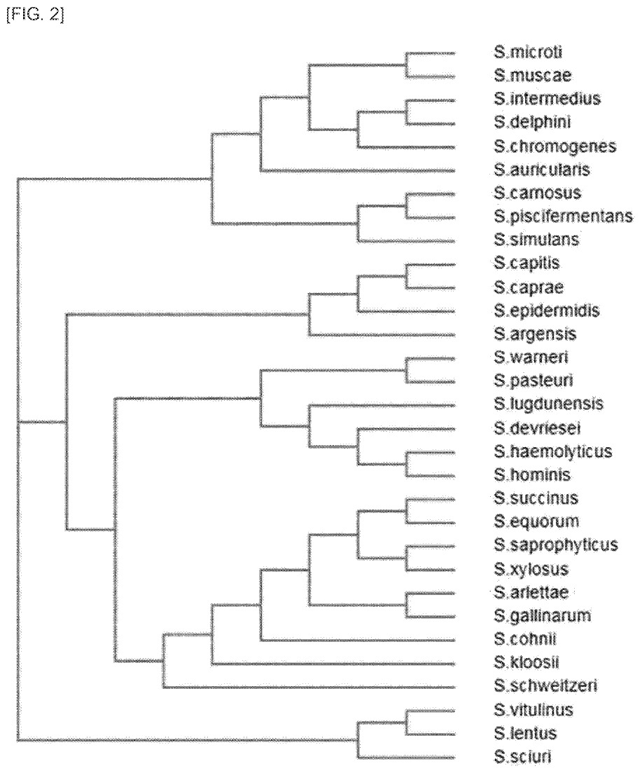

As a result, it has been confirmed that the homology of 16s rRNA sequences of the microorganisms belonging to the genus Staphylococcus known as nonpathogenic microorganisms between the strains is 92.78% or more in all cases (FIG. 2). The homology of the 31 microorganisms belonging to the genus Staphylococcus was in most cases 95% or more, and the homology was 92.78% to 95.21% in the case of Staphylococcus argensis.

This is a result supporting that the microorganisms belonging to the genus Staphylococcus classified as nonpathogenic microorganisms all have high genetic connectivity.

### Example 2-2: Confirmation of 16S rRNA Similarity of 20 Nonpathogenic Microorganisms Belonging to Genus Staphylococcus

The sequence homology was analyzed using the Clustal omega program (https://www.ebi.ac.uk/Tools/msa/clustalo) based on the 16s rRNA sequences of the 20 microorganisms belonging to the genus Staphylococcus of which the ability to produce allulose was confirmed in Example 1 above. The representative 16s rRNA sequences of the respective strains were confirmed via the NCBI database.

As a result, it has been confirmed that the homology of 16s rRNA sequences of the 20 microorganisms belonging to the genus Staphylococcus of which the ability to produce allulose has been confirmed is 95.97% or more.

From the Examples above, it has been confirmed that nonpathogenic microorganisms belonging to the genus Staphylococcus all have high genetic connectivity between species, and the microorganisms belonging to the genus Staphylococcus that have such high genetic connectivity exhibit the activity of producing allulose from fructose.

Based on the above description, it will be understood by those skilled in the art that the present disclosure may be implemented in a different specific form without changing the technical spirit or essential characteristics thereof.

Therefore, it should be understood that the above embodiment is not limitative, but illustrative in all aspects. The scope of the disclosure is defined by the appended claims rather than by the description preceding them, and therefore all changes and modifications that fall within metes and bounds of the claims or equivalents of such metes and bounds are therefore intended to be embraced by the claims.

1. A composition for allulose production comprising a microorganism belonging to the genus Staphylococcus or a culture of the microorganism belonging to the genus Staphylococcus.

2. The composition according to claim 1, wherein the microorganism belonging to the genus Staphylococcus is any one selected from the group consisting of Staphylococcus agnetis, Staphylococcus argensis, Staphylococcus argenteus, Staphylococcus adettae, Staphylococcus aureus, Staphylococcus auricularis, Staphylococcus capitis, Staphylococcus caprae, Staphylococcus camosus, Staphylococcus chromogenes, Staphylococcus cohnii, Staphylococcus condiment, Staphylococcus comubiensis, Staphylococcus delphini, Staphylococcus devriesei, Staphylococcus edaphicus, Staphylococcus epidermidis, Staphylococcus equorum, Staphylococcus faecalis, Staphylococcus felis, Staphylococcus fleurettii, Staphylococcus gallinarum, Staphylococcus haemolyticus, Staphylococcus hominis, Staphylococcus hyicus, Staphylococcus intermedius, Staphylococcus kloosii, Staphylococcus leei, Staphylococcus lentus, Staphylococcus lugdunensis, Staphylococcus lutrae, Staphylococcus lyticans, Staphylococcus massiliensis, Staphylococcus microti, Staphylococcus muscae, Staphylococcus nepalensis, Staphylococcus pasteuri, Staphylococcus petrasii, Staphylococcus pettenkoferi, Staphylococcus piscifermentans, Staphylococcus pseudintermedius, Staphylococcus pseudolugdunensis, Staphylococcus rostri, Staphylococcus saccharolyticus, Staphylococcus saprophyticus, Staphylococcus schleifer, Staphylococcus schweitzeri, Staphylococcus sciuri, Staphylococcus simiae, Staphylococcus simulans, Staphylococcus stepanovicii, Staphylococcus succinus, Staphylococcus vitulinus, Staphylococcus warneri, and Staphylococcus xylosus.

3. The composition according to claim 1, wherein the microorganism belonging to the genus Staphylococcus is any one selected from the group consisting of Staphylococcus carnosus, Staphylococcus xylosus, Staphylococcus vitulinus, Staphylococcus epidermidis, Staphylococcus warneri, Staphylococcus haemolyticus, Staphylococcus intermedius, Staphylococcus saprophyticus, Staphylococcus cohnii, Staphylococcus muscae, Staphylococcus lentus, Staphylococcus chromogenes, Staphylococcus caprae, Staphylococcus auricularis, Staphylococcus gallinarum, Staphylococcus arlettae, Staphylococcus equorum, Staphylococcus kloosii, Staphylococcus delphini, and Staphylococcus pasteuri.

4. The composition according to claim 1, wherein the microorganism belonging to the genus Staphylococcus is nonpathogenic.

5. A method for preparing allulose, the method comprising bringing a microorganism belonging to the genus Staphylococcus or a culture of the microorganism belonging to the genus Staphylococcus into contact with fructose to convert the fructose to allulose.

6. The preparation method according to claim 5, wherein the microorganism belonging to the genus Staphylococcus is any one selected from the group consisting of Staphylococcus agnetis, Staphylococcus argensis, Staphylococcus argenteus, Staphylococcus arlettae, Staphylococcus aureus, Staphylococcus auricularis, Staphylococcus capitis, Staphylococcus caprae, Staphylococcus camosus, Staphylococcus chromogenes, Staphylococcus cohnii, Staphylococcus condiment, Staphylococcus cornubiensis, Staphylococcus delphini, Staphylococcus devriesei, Staphylococcus edaphicus, Staphylococcus epidermidis, Staphylococcus equorum, Staphylococcus faecalis, Staphylococcus felis, Staphylococcus fleurettii, Staphylococcus gallinarum, Staphylococcus haemolyticus, Staphylococcus hominis, Staphylococcus hyicus, Staphylococcus intermedius, Staphylococcus kloosii, Staphylococcus leei, Staphylococcus lentus, Staphylococcus lugdunensis, Staphylococcus lutrae, Staphylococcus lyticans, Staphylococcus massiliensis, Staphylococcus microti, Staphylococcus muscae, Staphylococcus nepalensis, Staphylococcus pasteuri, Staphylococcus petrasii, Staphylococcus pettenkoferi, Staphylococcus piscifermentans, Staphylococcus pseudintermedius, Staphylococcus pseudolugdunensis, Staphylococcus rostri, Staphylococcus saccharolyticus, Staphylococcus saprophyticus, Staphylococcus schleiferi, Staphylococcus schweitzeri, Staphylococcus sciuri, Staphylococcus simiae, Staphylococcus simulans, Staphylococcus stepanovicii, Staphylococcus succinus, Staphylococcus vitulinus, Staphylococcus warneri, and Staphylococcus xylosus.

7. The preparation method according to claim 5, wherein the microorganism belonging to the genus Staphylococcus is any one selected from the group consisting of Staphylococcus carnosus, Staphylococcus xylosus, Staphylococcus vitulinus, Staphylococcus epidermidis, Staphylococcus warneri, Staphylococcus haemolyticus, Staphylococcus intermedius, Staphylococcus saprophyticus, Staphylococcus cohnii, Staphylococcus muscae, Staphylococcus lentus, Staphylococcus chromogenes, Staphylococcus caprae, Staphylococcus auricularis, Staphylococcus gallinarum, Staphylococcus arlettae, Staphylococcus equorum, Staphylococcus kloosii, Staphylococcus delphini, and Staphylococcus pasteuri.

8. The preparation method according to claim 5, which further comprises recovering converted allulose.

9. The preparation method according to claim 5, wherein the converting fructose to allulose is performed at a temperature of 40° C. to 70° C.

10. Use of a microorganism belonging to the genus Staphylococcus or a culture of the microorganism belonging to the genus Staphylococcus for allulose production.
